(19) United States
(12) Patent Application Publication
(10) Pub. No.: US 2026/0200883 A1
(43) Pub. Date: Jul. 16, 2026

(54) INTEGRATED MULTI-UNIT METHOD FOR CO-PRODUCTION OF 2,5-DIFORMYLFURAN AND 2,5-FURANDICARBOXYLIC ACID BASED ON BIPHASIC SOLVENT SYSTEM

(71) Applicant: DALIAN UNIVERSITY OF TECHNOLOGY, Dalian (CN)

(72) Inventors: Chang YU, Dalian (CN); Ji WEN, Dalian (CN); Fanshuo KONG, Dalian (CN); Jieshan QIU, Dalian (CN)

(21) Appl. No.: 19/563,417

(22) Filed: Mar. 11, 2026

(30) Foreign Application Priority Data
Oct. 20, 2025 (CN) 202511492494.4

(51) Int. Cl. C07D 307/68; C07D 307/46

(52) U.S. Cl. CPC C07D 307/68; C07D 307/46

(57) ABSTRACT

An integrated multi-unit method for co-production of 2,5-diformylfuran and 2,5-furandicarboxylic acid based on a biphasic solvent system belongs to the technical field of energy chemical engineering. First, 5-hydroxymethylfurfural enters a feedstock pretreatment unit, then forms a reaction solution with a stable water-organic solvent interface, passes through a reaction unit to generate 2,5-diformylfuran, 2,5-furandicarboxylic acid and intermediate oxidation products, and then enters a separation unit to achieve the separation of an organic solvent from an aqueous phase, and the separation of 2,5-diformylfuran from the organic solvent; the separated aqueous phase enters the reaction unit through a recycling unit to oxidize the intermediate oxidation product to 2,5-furandicarboxylic acid; the separation of an amphiphilic catalyst from the aqueous phase, and the separation of 2,5-furandicarboxylic acid from the aqueous phase are achieved; and finally, recovery is performed. The present invention is simple and controllable, has high substrate solubility and catalytic activity.

### TECHNICAL FIELD

The present invention belongs to the technical field of energy chemical engineering and biochemical industry, and relates to an integrated multi-unit method for co-production of 2,5-diformylfuran and 2,5-furandicarboxylic acid based on a biphasic solvent system.

### BACKGROUND

Biomass-derived 2,5-diformylfuran and 2,5-furandicarboxylic acid, which are important platform chemicals for furan-based products, can serve as intermediates for high-value-added compounds such as pharmaceuticals, antimicrobial agents, fluorescent agents and specialty adhesives. 2,5-Diformylfuran has a symmetrical molecular structure, is a highly regarded new energy chemical, and is widely used in battery separators, solid-state battery electrolytes, and electronic optical devices. The downstream product, 2,5-furandicarboxylic acid with a unique rigid structure, can replace traditional petroleum-based terephthalic acid, and is recognized by the U.S. Department of Energy as one of the 12 high-value-added bio-based chemicals.

The oxidation of 5-hydroxymethylfurfural to prepare 2,5-diformylfuran and 2,5-furandicarboxylic acid is the most extensively research route. The synthesis of 2,5-diformylfuran is typically carried out in an organic solvent. The non-polar organic solvent with a low boiling point can prevent over-oxidation of the target product. However, 5-hydroxymethylfurfural has low solubility and slow conversion rate in this solvent. Water is a primary solvent for synthesizing 2,5-furandicarboxylic acid, with high solubility for 5-hydroxymethylfurfural. However, at high concentration, 5-hydroxymethylfurfural is prone to self-polymerization and Cannizzaro disproportionation reactions in the water, which reduces product yield. To address these problems, domestic and foreign scholars have conducted extensive research and proposed some solutions. For example:

Chinese invention patent (CN202311362580) provides a method for directly preparing 2,5-diformylfuran from carbohydrate compounds. In a biphasic system composed of water and an organic solvent, a catalyst component dissolved in an aqueous phase is used for catalytic conversion of carbohydrate compounds to generate 5-hydroxymethylfurfural, and then the catalyst component dispersed in an organic phase is used for preparing 2,5-diformylfuran. However, the method is highly dependent on the precise design of a catalyst, and in the preparation process of the catalyst, an explosive strong oxidant, potassium permanganate, needs to be used, which increases the operational risks and leads to the generation of manganese-containing wastewater. Chinese invention patent (CN201680072169) provides a method for preparing 2,5-furandicarboxylic acid. First, partial oxidation of 5-hydroxymethylfurfural is achieved in a mixed solution of water and an organic solvent with a high boiling point, subsequently, a water-miscible organic solvent with a high boiling point is extracted using an organic solvent with a low boiling point, and finally, the partially oxidized product is completely oxidized to 2,5-furandicarboxylic acid. However, this method requires different catalysts for the two reaction steps and involves the use of expensive Pt-based catalysts. Meanwhile, the organic solvent with a low boiling point is introduced as an extractant. After the reaction is completed, an alkaline reaction solution needs to be subjected to acid treatment to obtain a target product, which significantly increases the difficulty of separating and recovering the solvent, the catalysts and 2,5-furandicarboxylic acid.

In addition, the oxidation of 5-hydroxymethylfurfural to produce 2,5-diformylfuran has a low energy barrier and high reaction rate, but the side chain of the formyl groups in 2,5-diformylfuran is prone to over oxidation; and the synthesis of 2,5-furandicarboxylic acid needs to overcome a higher reaction energy barrier, and exhibits slow reaction kinetics. The kinetic and thermodynamic mismatching of the synthesis of 2,5-diformylfuran and 2,5-furandicarboxylic acid makes it difficult to prepare both products simultaneously in a single system. Therefore, it is of great importance to develop an integrated multi-unit system with high substrate solubility and simple separation and recovery features to overcome kinetic and thermodynamic limitations to achieve the co-production of 2,5-diformylfuran and 2,5-furandicarboxylic acid.

### SUMMARY

To solve the above problems, the present invention provides an integrated multi-unit method for co-production of 2,5-diformylfuran and 2,5-furandicarboxylic acid based on a biphasic solvent system. The present invention has the advantages of simple process, high substrate solubility, the capability of co-production of 2,5-diformylfuran and 2,5-furandicarboxylic acid, and the recyclability and recoverability of amphiphilic catalysts and organic solvents.

To achieve the above purpose, the present invention adopts the following technical solution:

An integrated multi-unit method for co-production of 2,5-diformylfuran and 2,5-furandicarboxylic acid based on a biphasic solvent system is provided, which is implemented on the basis of a feedstock pretreatment unit, a reaction unit, a separation unit, a recycling unit and a recovery unit; and multi-unit integration refers to the integration of the feedstock pretreatment unit, the reaction unit, the separation unit, the recycling unit and the recovery unit. In the integrated multi-unit method, first, 5-hydroxymethylfurfural enters the feedstock pretreatment unit, then forms a reaction solution with a stable water-organic solvent interface, passes through the reaction unit to generate 2,5-diformylfuran, 2,5-furandicarboxylic acid and intermediate oxidation products,and then enters the separation unit to achieve the separation of an organic solvent from an aqueous phase, and the separation of 2,5-diformylfuran from the organic solvent in sequence, wherein the organic solvent contains 2,5-diformylfuran and the aqueous phase contains an amphiphilic catalyst, 2,5-furandicarboxylic acid and intermediate oxidation products; the separated aqueous phase reenters the reaction unit through the recycling unit until the intermediate oxidation products are completely oxidized to 2,5-furandicarboxylic acid, and subsequently, the separation of the amphiphilic catalyst from the aqueous phase, and the separation of 2,5-furandicarboxylic acid from the aqueous phase are achieved in the separation unit; and the separated organic solvent can pass through the recycling unit for a next reaction and is recovered after the reaction is completed. Finally, the organic solvent, 2,5-diformylfuran, 2,5-furandicarboxylic acid and the amphiphilic catalyst are obtained in the recovery unit. The method comprises the following steps:

step 1: preparing the reaction solution with a stable water-organic solvent interface, comprising the following specific steps:

step 1.1: preparing a surface-modified carbon nanotube;

at room temperature, adding the carbon nanotube to a mixed strong acid, stirring for 0.5-2 h, and continuing to stir in an oil bath at 25-90 ^{o}C for 2-12 h to obtain dispersion liquid; diluting the dispersion liquid, filtering and washing with water for several times to obtain precipitate; and drying the obtained precipitate at 50-90 ^{o}C for 6-24 h to obtain the surface-modified carbon nanotube. In the step, abundant hydrophilic oxygen-containing functional groups such as hydroxyl and carboxyl are introduced onto a hydrophobic surface of the carbon nanotube, which significantly enhances the capability to stabilize the water-organic solvent interface.

Further, the mixed strong acid is a mixed solution of two strong acids, the strong acids are one of hydrochloric acid, sulfuric acid and nitric acid, and the volume ratio of the two strong acids is 3: 1 to 1:3; and 0.5-4 g of carbon nanotube is added correspondingly to every 200 mL of the mixed strong acid.

Step 1.2: preparing the amphiphilic catalyst;

Step 1.2.1: at room temperature, adding the surface-modified carbon nanotube to a metal salt solution, performing ultrasonic treatment for 0.5-10 min and stirring for 6-24 h to obtain a mixed solution. In the step, the surface-modified carbon nanotube achieves the anchoring of active metals through electrostatic adsorption and chemical bonding effects.

Further, in the metal salt solution, a solvent is one of water, toluene and cyclohexane, a solute is metal salt, and the metal salt is one of palladium acetate, ruthenium chloride, palladium chloride, chloroplatinic acid, ruthenium acetylacetonate and platinum bis(acetylacetonate). 0.2-3 g of the surface-modified carbon nanotube and 0.02-0.1 g of the metal salt are added correspondingly to every 50 mL of the solvent.

Step 1.2.2: drying the mixed solution at 50-90 ^{o}C for 6-24 h, placing the obtained solid powder into a hydrogen atmosphere and reducing at a heating rate of 1-5 ^{o}C/min at 100-400 ^{o}C for 1-4 h to obtain the amphiphilic catalyst. In the step, active metals not stably immobilized migrate or fall off. However, active metals anchored on the surface-modified carbon nanotube are reduced from a high-valence state to a low-valence state by hydrogen, thereby finally forming a stable and highly active catalytic center.

Step 1.3: building a stable water-organic solvent interface;

at room temperature, adding a mixture of 5-hydroxymethylfurfural, the amphiphilic catalyst obtained in step 1.2, water and the organic solvent to the feedstock pretreatment unit and processing for 2-10 min to obtain the reaction solution with the stable water-organic solvent interface. In the step, the aggregates of the amphiphilic catalyst and 5-hydroxymethylfurfural are broken up, the water and the organic solvent are fully mixed, and the water-organic solvent interface is stabilized by the uniformly dispersed amphiphilic catalyst.

Further, the amphiphilic catalyst can simultaneously serve as an emulsifier to form micron-sized Pickering emulsion droplets to stabilize the water-organic solvent interface.

Further, the organic solvent is one of non-polar and water-immiscible organic matter with a boiling point lower than 180 ^{o}C, such as toluene, paraxylene, cyclohexane, n-hexane or ethyl acetate. The volume ratio of the water to the organic solvent is 0.1-9: 1; and the mass ratio of the amphiphilic catalyst to 5-hydroxymethylfurfural is 0.1-1: 1.

Further, the feedstock pretreatment unit comprises an emulsification device, and the emulsification device is one of an ultrasonic emulsifier, a high speed stirrer, a high pressure homogenizing emulsifier, and a static mixer.

Step 2: synthesizing 2,5-diformylfuran and recovering the organic solvent, comprising the following specific steps:

Step 2.1: adding the reaction solution, obtained in step 1, which has a stable water-organic solvent interface, to a high pressure batch reactor, purging the oxygen for 1-3 times, subsequently filling with 0.1-2 MPa of oxygen, heating to 80-160 ^{o}C, reacting for 0.5-5 h, and then cooling to room temperature; processing the reaction solution in a deemulsification device at a rotational speed of 1000-10000 rpm for 1-10 min to achieve the stratification of the water and the organic solvent to complete a first reaction, wherein the organic solvent is located in an upper layer and a lower layer is the aqueous phase. The 2,5-diformylfuran is dissolved in the organic solvent, the amphiphilic catalyst, 2,5-furandicarboxylic acid and the intermediate oxidation products are dispersed in the water, and the yield of the product can be determined by high performance liquid chromatography (HPLC). In the step, the water, which serves as a polar solvent, can enhance the solubility of 5-hydroxymethylfurfural to facilitate the conversion of 5-hydroxymethylfurfural and the production of 2,5-furandicarboxylic acid, and the non-polar organic solvent with a low boiling point can inhibit side reactions to facilitate the production of 2,5-diformylfuran and reduce the difficulty of product separation. In addition, driven by the difference in solubility, hydrophilic products (such as 2,5-furandicarboxylic acid) and hydrophobic products (such as 2,5-diformylfuran) spontaneously transfer into the water and the organic solvent.

Step 2.2: repeating step 1.3 and step 2.1, and conducting circular reactions for 1-5 times. Before each circular reaction begins, the upper organic solvent containing 2,5-diformylfuran needs to be transferred and collected at first; subsequently, the same mass of 5-hydroxymethylfurfural and the same mass of organic solvent as those in the first reaction are replenished to the lower water phase; and the mixed solution is delivered to the feedstock pretreatment unit to repeat step 1.3 and step 2.1.

Step 2.3: after the circular reactions are completed, transferring and collecting the upper organic solvent again; subsequently, transferring the obtained organic solvent enriched with 2,5-diformylfuran to a vacuum distillation device; and performing separation under a pressure of 5-50 kPa and a temperature of 50-100 ^{o}C to obtain the lower aqueous phase and the upper organic solvent. The separated organic solvent can be recycled in the reaction unit; and after all the steps are completed, the organic solvent can be recovered together with 2,5-diformylfuran.

Step 3: synthesizing 2,5-furandicarboxylic acid and recovering the amphiphilic catalyst, comprising the following specific steps:

Step 3.1: adding the remaining lower aqueous phase after the separation from step 2.3 to the high pressure batch reactor, purging oxygen for 1-3 times, subsequently filling with 0.1-2 MPa of oxygen, heating to 80-160 ^{o}C again, reacting for 12-36 h, and completely oxidizing the intermediate oxidation products into 2,5-furandicarboxylic acid to obtain the reaction solution.

Step 3.2: processing the reaction solution at a rotational speed of 5000-18000 rpm in a liquid-solid separation device for 5-15 min to obtain the amphiphilic catalyst and an aqueous solution enriched with 2,5-furandicarboxylic acid; transferring the aqueous solution to a cooling crystallization device, cooling and crystallizing at -30 to 0 ^{o}C, and separating through a filtration device to obtain 2,5-furandicarboxylic acid.

Further, the feedstock pretreatment unit, the reaction unit, the separation unit, the recycling unit and the recovery unit adopted by the present invention are specifically as follows: the feedstock pretreatment unit comprises the emulsification device, and the emulsification device is in communication with the reaction unit through a liquid-solid conveying pipeline; the reaction unit is provided with the high pressure batch reactor, the materials passing through the feedstock pretreatment unit undergo chemical reactions in the high pressure batch reactor, and the materials after the reactions are completed pass through the liquid-solid conveying pipeline and enter the separation unit; the separation unit comprises the deemulsification device, the distillation device, a liquid-solid phase separation device, the cooling crystallization device and the filtration device; the top of the deemulsification device is in communication with the distillation device through a liquid conveying pipeline, and the bottom is connected to the liquid-solid phase separation device through the liquid-solid conveying pipeline; the liquid-solid phase separation device is connected to the cooling crystallization device through the liquid conveying pipeline; and the cooling crystallization device is in communication with the filtration device through the liquid-solid conveying pipeline. The materials passing through the separation unit can enter the recycling unit and the recovery unit through the liquid-solid conveying pipeline.

The present invention has the operating principle that:

Under the combined action of the emulsification device and the amphiphilic catalyst, the reaction solution passes through the feedstock pretreatment unit and then forms the stable water-organic solvent interface. Subsequently, in the reaction unit, water, which serves as a polar solvent, can increase the solubility of 5-hydroxymethylfurfural and facilitate the synthesis of 2,5-furandicarboxylic acid; and the non-polar organic solvent with a low boiling point can facilitate the synthesis of 2,5-diformylfuran and simplify the separation process. The hydrophobic 2,5-diformylfuran is enriched in the organic solvent, and the hydrophilic 2,5-furandicarboxylic acid and the intermediate oxidation products are enriched in the water. Then, the organic solvent is transferred in time through the separation unit, which kinetically prevents the generated 2,5-diformylfuran from being over-oxidized, and moreover, the separated organic solvent can be used by the recycling unit for a next reaction; The aqueous phase containing the amphiphilic catalyst can also increase the processing capacity of 5-hydroxymethylfurfural by recycling, and in the subsequent reaction, the intermediate oxidation products can be completely oxidized to 2,5-furandicarboxylic acid. Finally, in the recovery unit, the organic solvent, 2,5-diformylfuran, 2,5-furandicarboxylic acid and the amphiphilic catalyst are obtained.

Compared with the prior art, the present invention has the following beneficial effects:

(1) Through the construction of the biphasic solvent system and the integration of multiple unit operations, the present invention increases the solubility of 5-hydroxymethylfurfural, overcomes thermodynamic and kinetic limitations, and achieves the co-production of 2,5-diformylfuran and 2,5-furandicarboxylic acid, wherein the mole fraction of 2,5-diformylfuran in the organic solvent can be up to 98%, and the mole fraction of 2,5-furandicarboxylic acid in the aqueous phase can be up to 94%.

(2) Compared with pure water and pure toluene systems without integrated units, the total yields of 2,5-diformylfuran and 2,5-furandicarboxylic acid after 4 cycles in the present invention are 2.2 times that of the pure toluene system and 3.3 times that of the pure water system.

(3) Through simple separation operation, the present invention can obtain 2,5-diformylfuran and 2,5-furandicarboxylic acid, and can also efficiently recover the organic solvent and the amphiphilic catalyst, with recovery rates being 85% and 95% respectively.

### DESCRIPTION OF DRAWINGS

FIG. 1 is a flow chart of an integrated multi-unit method of the present invention;

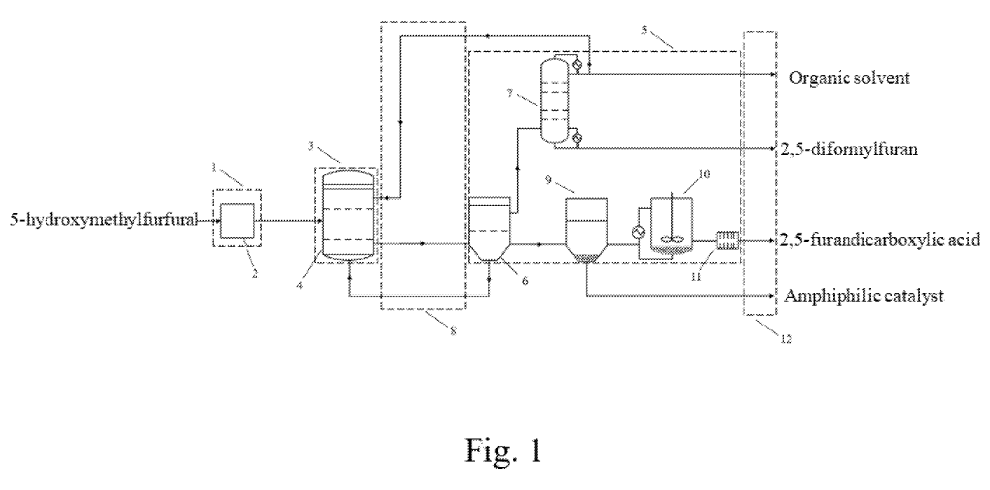

FIG. 2 is a comparison diagram of yields of 2,5-diformylfuran and 2,5-furandicarboxylic acid of embodiment 3 with reference example 1 and reference example 2.

FIG. 3 is a comparison diagram of product distribution of embodiment 3 with reference example 1 and reference example 2.

In the figures: 1 feedstock pretreatment unit, 2 emulsification device, 3 reaction unit, 4 high pressure batch reactor, 5 separation unit, 6 deemulsification device, 7 distillation device, 8 recycling unit, 9 liquid-solid phase separation device, 10 cooling crystallization device, 11 filtration device, and 12 recovery unit.

### DETAILED DESCRIPTION

In view of the numerous deficiencies in the prior art, the inventor of this case proposes the technical solution of the present invention after long-term research and extensive practice. However, it should be understood that each of the above technical features of the present invention and each of the technical features specifically described in the following (embodiments) may be combined with each other within the scope of the present invention, thereby constituting a new or preferred technical solution. The present invention will be further described below in combination with embodiments.

An integrated multi-unit method for co-production of 2,5-diformylfuran and 2,5-furandicarboxylic acid based on a biphasic solvent system is implemented on the basis of a feedstock pretreatment unit 1, a reaction unit 3, a separation unit 5, a recycling unit 8 and a recovery unit 12; specifically, the feedstock pretreatment unit 1 comprises an emulsification device, and the emulsification device 2 is in communication with the reaction unit 3 through a liquid-solid conveying pipeline; the reaction unit 3 is provided with a high pressure batch reactor 4, materials passing through the feedstock pretreatment unit 1 undergo chemical reactions in the high pressure batch reactor 4, and the materials after the reactions are completed pass through the liquid-solid conveying pipeline and enter the separation unit 5; the separation unit 5 comprises a deemulsification device 6, a distillation device 7, a liquid-solid phase separation device 9, a cooling crystallization device 10 and a filtration device 11; the top of the deemulsification device 6 is in communication with the distillation device 7 through a liquid conveying pipeline, and the bottom is connected to the liquid-solid phase separation device 9 through the liquid-solid conveying pipeline; the liquid-solid phase separation device 9 is connected to the cooling crystallization device 10 through the liquid conveying pipeline; and the cooling crystallization device 10 is in communication with the filtration device 11 through the liquid-solid conveying pipeline. The materials passing through the separation unit 5 can enter the recycling unit 8 and the recovery unit 12 through the liquid-solid conveying pipeline.

### Embodiment 1

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 2.5 g of a carbon nanotube was added to a mixed solution of 100 mL of hydrochloric acid and 100 mL of sulfuric acid at room temperature and stirred for 1.5 h. Then, the solution was stirred in an oil bath at 25 ^{o}C for 12 h to obtain dispersion liquid. The dispersion liquid was diluted, filtered and washed with water for several times to obtain precipitate. The obtained precipitate was dried at 60 ^{o}C for 24 h to obtain a surface-modified carbon nanotube.

Step 1.2: first, at room temperature, the surface-modified carbon nanotube was added to 40.3 mL of 1 mg mL⁻¹ platinum bis(acetylacetonate) toluene solution, subjected to ultrasonic treatment for 8 min and stirred for 10 h to obtain a mixed solution. Then, the mixed solution was dried at 60 ^{o}C for 24 h, and the obtained solid powder was placed into a hydrogen atmosphere, and reduced at a heating rate of 3 ^{o}C/min at 150 ^{o}C for 3 h to obtain an amphiphilic catalyst.

Step 1.3: at room temperature, a mixture of 80 mg of 5-hydroxymethylfurfural, 30 mg of the amphiphilic catalyst, 15 mL of water and 5 mL of n-hexane was added to an ultrasonic emulsifier and processed for 8 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen once, subsequently filled with 0.3 MPa of oxygen, heated to 80 ^{o}C, reacted for 4 h, and then cooled to room temperature. The reaction solution was processed in a centrifuge at a rotational speed of 3000 rpm for 10 min to achieve the stratification of the water and the n-hexane to complete a first reaction, wherein the organic solvent was located in an upper layer and contained the synthesized 2,5-diformylfuran, and a lower layer was the aqueous phase. In the present embodiment, 2,5-diformylfuran was dissolved in the n-hexane, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted twice. Before each circular reaction began, the upper n-hexane containing 2,5-diformylfuran needed to be transferred and collected at first; subsequently, 80 mg of 5-hydroxymethylfurfural and 5 mL of n-hexane were replenished to the lower water phase; and the mixed solution was added to the ultrasonic emulsifier to repeat step 1.3 and step 2.1.

Step 2.3: after the circular reactions were completed, the upper n-hexane was transferred and collected again; subsequently, the obtained n-hexane enriched with 2,5-diformylfuran was transferred to a vacuum distillation device; and separation was performed at 30 kPa and 80 ^{o}C. The separated n-hexane can be recycled in the reaction unit. After all the experimental steps were completed, the n-hexane can be recovered together with 2,5-diformylfuran. The conversion rate of 5-hydroxymethylfurfural was 100%, the mole fraction of 2,5-diformylfuran in the n-hexane was 96%, and the recovery rate of the n-hexane was 84%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen once, subsequently filled with 0.3 MPa of oxygen, heated to 80 ^{o}C, and reacted for 20 h, and the intermediate product was completely oxidized into 2,5-furandicarboxylic acid to obtain the reaction solution.

Step 3.2: the reaction solution was processed at a rotational speed of 5000 rpm in the centrifuge for 15 min to obtain the amphiphilic catalyst and an aqueous solution enriched with 2,5-furandicarboxylic acid. The aqueous solution was transferred to a cooling crystallization device, cooled and crystallized at -30 ^{o}C, and separated through a filtration device to obtain 2,5-furandicarboxylic acid. The mole fraction of 2,5-furandicarboxylic acid in the water was 87%, the recovery rate of the amphiphilic catalyst was 81%, and the total yield of 2,5-diformylfuran and 2,5-furandicarboxylic acid was 0.73 mmol.

### Embodiment 2

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 1.5 g of a carbon nanotube was added to a mixed solution of 100 mL of sulphuric acid and 100 mL of nitric acid at room temperature and stirred for 2 h. Then, the solution was stirred in an oil bath at 70 ^{o}C for 8 h to obtain dispersion liquid. The dispersion liquid was diluted, filtered and washed with water for several times to obtain precipitate. The obtained precipitate was dried at 90 ^{o}C for 10 h to obtain a surface-modified carbon nanotube.

Step 1.2: first, at room temperature, the surface-modified carbon nanotube was added to 42.2 mL of 1 mg mL⁻¹ palladium acetate cyclohexane solution, subjected to ultrasonic treatment for 3 min and stirred for 8 h to obtain a mixed solution. Then, the mixed solution was dried at 90 ^{o}C for 10 h, and the obtained solid powder was placed into a hydrogen atmosphere, and reduced at a heating rate of 1 ^{o}C/min at 200 ^{o}C for 2.5 h to obtain an amphiphilic catalyst.

Step 1.3: at room temperature, a mixture of 200 mg of 5-hydroxymethylfurfural, 50 mg of the amphiphilic catalyst, 18 mL of water and 2 mL of ethyl acetate was added to a static mixer and processed for 3 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen twice, subsequently filled with 0.5 MPa of oxygen, heated to 140 ^{o}C, reacted for 2 h, and then cooled to room temperature. The reaction solution was processed in a centrifuge at a rotational speed of 9000 rpm for 8 min to achieve the stratification of the water and the ethyl acetate to complete a first reaction, wherein the organic solvent was located in an upper layer and contained the synthesized 2,5-diformylfuran, and a lower layer was the aqueous phase. In the present embodiment, 2,5-diformylfuran was dissolved in the ethyl acetate, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted once. Before each circular reaction began, the upper ethyl acetate containing 2,5-diformylfuran needed to be transferred and collected at first; subsequently, 200 mg of 5-hydroxymethylfurfural and 2 mL of ethyl acetate were replenished to the lower water phase; and the mixed solution was added to the static mixer to repeat step 1.3 and step 2.1.

Step 2.3: after the circular reactions were completed, the upper ethyl acetate was transferred and collected again; subsequently, the obtained ethyl acetate enriched with 2,5-diformylfuran was transferred to a vacuum distillation device; and separation was performed at 40 kPa and 90 ^{o}C. The separated ethyl acetate can be recycled in the reaction unit. After all the experimental steps were completed, the ethyl acetate can be recovered together with 2,5-diformylfuran. The conversion rate of 5-hydroxymethylfurfural was 100%, the mole fraction of 2,5-diformylfuran in the ethyl acetate was 93%, and the recovery rate of the ethyl acetate was 81%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen twice, subsequently filled with 0.5 MPa of oxygen, heated to 140 ^{o}C, and reacted for 30 h, and the intermediate product was completely oxidized into 2,5-furandicarboxylic acid to obtain the reaction solution.

Step 3.2: the reaction solution was processed at a rotational speed of 15000 rpm in the centrifuge for 8 min to obtain the amphiphilic catalyst and an aqueous solution enriched with 2,5-furandicarboxylic acid. The aqueous solution was transferred to a cooling crystallization device, cooled and crystallized at -15 ^{o}C, and separated through a filtration device to obtain 2,5-furandicarboxylic acid. The mole fraction of 2,5-furandicarboxylic acid in the water was 97%, the recovery rate of the amphiphilic catalyst was 96%, and the total yield of 2,5-diformylfuran and 2,5-furandicarboxylic acid was 0.39 mmol.

### Embodiment 3

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 2 g of a carbon nanotube was added to a mixed solution of 50 mL of nitric acid and 150 mL of sulfuric acid at room temperature and stirred for 0.5 h. Then, the solution was stirred in an oil bath at 60 ^{o}C for 5 h to obtain dispersion liquid. The dispersion liquid was diluted, filtered and washed with water for several times to obtain precipitate. The obtained precipitate was dried at 80 ^{o}C for 12 h to obtain a surface-modified carbon nanotube.

Step 1.2: first, at room temperature, the surface-modified carbon nanotube was added to 25 mL of 1 mg mL⁻¹ aqueous ruthenium chloride solution, subjected to ultrasonic treatment for 0.5 min and stirred for 12 h to obtain a mixed solution. Then, the mixed solution was dried at 80 ^{o}C for 12 h, and the obtained solid powder was placed into a hydrogen atmosphere, and reduced at a heating rate of 3 ^{o}C/min at 250 ^{o}C for 2 h to obtain an amphiphilic catalyst.

Step 1.3: at room temperature, a mixture of 50 mg of 5-hydroxymethylfurfural, 30 mg of the amphiphilic catalyst, 10 mL of water and 10 mL of toluene was added to an ultrasonic emulsifier and processed for 5 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen for three times, subsequently filled with 1 MPa of oxygen, heated to 130 ^{o}C, reacted for 3 h, and then cooled to room temperature. The reaction solution was processed in a centrifuge at a rotational speed of 5000 rpm for 1 min to achieve the stratification of the water and the toluene to complete a first reaction, wherein the organic solvent was located in an upper layer and contained the synthesized 2,5-diformylfuran, and a lower layer was the aqueous phase. In the present embodiment, 2,5-diformylfuran was dissolved in the toluene, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted for three times. Before each circular reaction began, the upper toluene containing 2,5-diformylfuran needed to be transferred and collected at first; subsequently, 50 mg of 5-hydroxymethylfurfural and 10 mL of toluene were replenished to the lower water phase; and the mixed solution was added to the ultrasonic emulsifier to repeat step 1.3 and step 2.1.

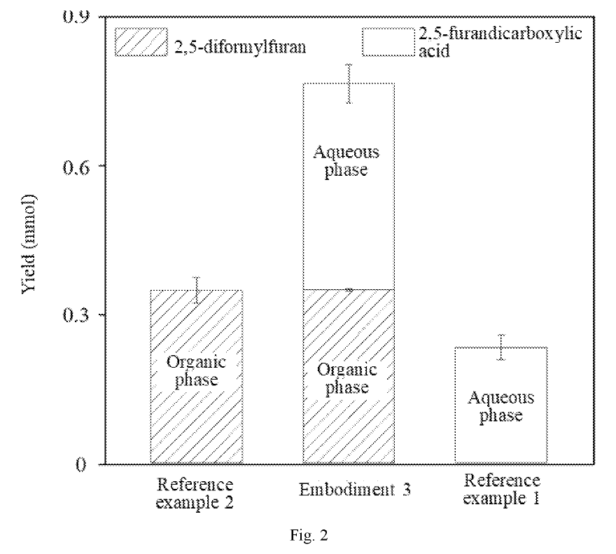

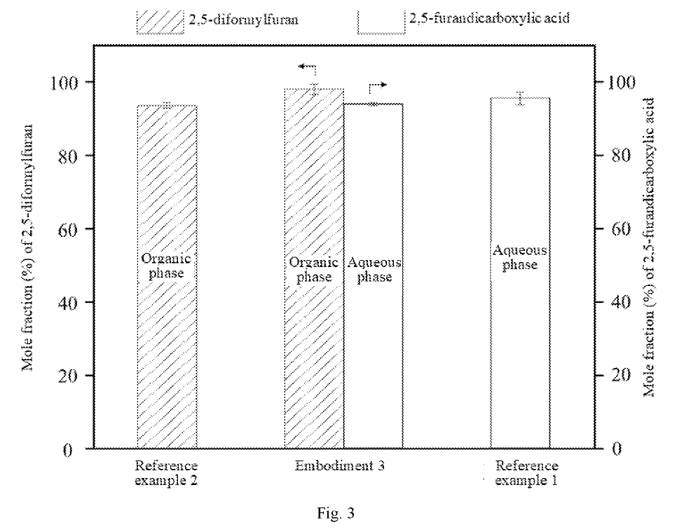

Step 2.3: after the circular reactions were completed, the upper toluene was transferred and collected again; subsequently, the obtained toluene enriched with 2,5-diformylfuran was transferred to a vacuum distillation device; and separation was performed at 20 kPa and 60 ^{o}C. The separated toluene can be recycled in the reaction unit. After all the experimental steps were completed, the toluene can be recovered together with 2,5-diformylfuran. Results were shown in FIG. 2 and FIG. 3. The conversion rate of 5-hydroxymethylfurfural was 100%, the mole fraction of 2,5-diformylfuran in the toluene was 98%, and the recovery rate of the toluene was 85%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen for three times, subsequently filled with 1 MPa of oxygen, heated to 130 ^{o}C, and reacted for 12 h, and the intermediate product was completely oxidized into 2,5-furandicarboxylic acid to obtain the reaction solution.

Step 3.2: the reaction solution was processed at a rotational speed of 10000 rpm in the centrifuge for 5 min to obtain the amphiphilic catalyst and an aqueous solution enriched with 2,5-furandicarboxylic acid. The aqueous solution was transferred to a cooling crystallization device, cooled and crystallized at 0 ^{o}C, and separated through a filtration device to obtain 2,5-furandicarboxylic acid. Results were shown in FIG. 2 and FIG. 3. The mole fraction of 2,5-furandicarboxylic acid in the water was 94%, the recovery rate of the amphiphilic catalyst was 95%, and the total yield of 2,5-diformylfuran and 2,5-furandicarboxylic acid was 0.77 mmol.

### Embodiment 4

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 0.5 g of a carbon nanotube was added to a mixed solution of 100 mL of hydrochloric acid and 100 mL of sulphuric acid at room temperature and stirred for 2 h. Then, the solution was stirred in an oil bath at 30 ^{o}C for 6 h to obtain dispersion liquid. The dispersion liquid was diluted, filtered and washed with water for several times to obtain precipitate. The obtained precipitate was dried at 90 ^{o}C for 6 h to obtain a surface-modified carbon nanotube.

Step 1.2: first, at room temperature, the surface-modified carbon nanotube was added to 50 mL of 2 mg mL⁻¹ ruthenium acetylacetonate toluene solution, subjected to ultrasonic treatment for 10 min and stirred for 24 h to obtain a mixed solution. Then, the mixed solution was dried at 90 ^{o}C for 6 h, and the obtained solid powder was placed into a hydrogen atmosphere, and reduced at a heating rate of 2 ^{o}C/min at 100 ^{o}C for 4 h to obtain an amphiphilic catalyst.

Step 1.3: at room temperature, a mixture of 40 mg of 5-hydroxymethylfurfural, 40 mg of the amphiphilic catalyst, 5 mL of water and 15 mL of toluene was added to a high speed stirrer and processed for 10 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen twice, subsequently filled with 0.1 MPa of oxygen, heated to 160 ^{o}C, reacted for 0.5 h, and then cooled to room temperature. The reaction solution was processed in a centrifuge at a rotational speed of 10000 rpm for 2 min to achieve the stratification of the water and the toluene to complete a first reaction, wherein the organic solvent was located in an upper layer and contained the synthesized 2,5-diformylfuran, and a lower layer was the aqueous phase. In the present embodiment, 2,5-diformylfuran was dissolved in the toluene, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted twice. Before each circular reaction began, the upper toluene containing 2,5-diformylfuran needed to be transferred and collected at first; subsequently, 40 mg of 5-hydroxymethylfurfural and 15 mL of toluene were replenished to the lower water phase; and the mixed solution was added to the high speed stirrer to repeat step 1.3 and step 2.1.

Step 2.3: after the circular reactions were completed, the upper toluene was transferred and collected again; subsequently, the obtained toluene enriched with 2,5-diformylfuran was transferred to a vacuum distillation device; and separation was performed at 5 kPa and 50 ^{o}C. The separated toluene can be recycled in the reaction unit. After all the experimental steps were completed, the toluene can be recovered together with 2,5-diformylfuran. The conversion rate of 5-hydroxymethylfurfural was 100%, the mole fraction of 2,5-diformylfuran in the toluene was 91%, and the recovery rate of the toluene was 87%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen twice, subsequently filled with 0.1 MPa of oxygen, heated to 160 ^{o}C, and reacted for 15 h, and the intermediate product was completely oxidized into 2,5-furandicarboxylic acid to obtain the reaction solution.

Step 3.2: the reaction solution was processed at a rotational speed of 18000 rpm in the centrifuge for 6 min to obtain the amphiphilic catalyst and an aqueous solution enriched with 2,5-furandicarboxylic acid. The aqueous solution was transferred to a cooling crystallization device, cooled and crystallized at -10 ^{o}C, and separated through a filtration device to obtain 2,5-furandicarboxylic acid. The mole fraction of 2,5-furandicarboxylic acid in the water was 90%, the recovery rate of the amphiphilic catalyst was 97%, and the total yield of 2,5-diformylfuran and 2,5-furandicarboxylic acid was 0.38 mmol.

Embodiment 5

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 4 g of a carbon nanotube was added to a mixed solution of 150 mL of hydrochloric acid and 50 mL of nitric acid at room temperature and stirred for 1.5 h. Then, the solution was stirred in an oil bath at 90 ^{o}C for 2 h to obtain dispersion liquid. The dispersion liquid was diluted, filtered and washed with water for several times to obtain precipitate. The obtained precipitate was dried at 50 ^{o}C for 24 h to obtain a surface-modified carbon nanotube.

Step 1.2: first, at room temperature, the surface-modified carbon nanotube was added to 50 mL of 0.4 mg mL⁻¹ aqueous chloroplatinic acid solution, subjected to ultrasonic treatment for 2 min and stirred for 12 h to obtain a mixed solution. Then, the mixed solution was dried at 50 ^{o}C for 24 h, and the obtained solid powder was placed into a hydrogen atmosphere, and reduced at a heating rate of 5 ^{o}C/min at 400 ^{o}C for 1 h to obtain an amphiphilic catalyst.

Step 1.3: at room temperature, a mixture of 100 mg of 5-hydroxymethylfurfural, 10 mg of the amphiphilic catalyst, 2 mL of water and 18 mL of paraxylene was added to a high pressure homogenizing emulsifier and processed for 2 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen twice, subsequently filled with 2 MPa of oxygen, heated to 90 ^{o}C, reacted for 5 h, and then cooled to room temperature. The reaction solution was processed in a centrifuge at a rotational speed of 1000 rpm for 5 min to achieve the stratification of the water and the paraxylene to complete a first reaction, wherein the organic solvent was located in an upper layer and contained the synthesized 2,5-diformylfuran, and a lower layer was the aqueous phase. In the present embodiment, 2,5-diformylfuran was dissolved in the paraxylene, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted for four times. Before each circular reaction began, the upper paraxylene containing 2,5-diformylfuran needed to be transferred and collected at first; subsequently, 100 mg of 5-hydroxymethylfurfural and 18 mL of paraxylene were replenished to the lower water phase; and the mixed solution was added to the high pressure homogenizing emulsifier to repeat step 1.3 and step 2.1.

Step 2.3: after the circular reactions were completed, the upper paraxylene was transferred and collected again; subsequently, the obtained paraxylene enriched with 2,5-diformylfuran was transferred to a vacuum distillation device; and separation was performed at 50 kPa and 100 ^{o}C. The separated paraxylene can be recycled in the reaction unit. After all the experimental steps were completed, the paraxylene can be recovered together with 2,5-diformylfuran. The conversion rate of 5-hydroxymethylfurfural was 100%, the mole fraction of 2,5-diformylfuran in the paraxylene was 95%, and the recovery rate of the paraxylene was 82%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen twice, subsequently filled with 2 MPa of oxygen, heated to 90 ^{o}C, and reacted for 36 h, and the intermediate product was completely oxidized into 2,5-furandicarboxylic acid to obtain the reaction solution.

Step 3.2: the reaction solution was processed at a rotational speed of 16000 rpm in the centrifuge for 7 min to obtain the amphiphilic catalyst and an aqueous solution enriched with 2,5-furandicarboxylic acid. The aqueous solution was transferred to a cooling crystallization device, cooled and crystallized at -20 ^{o}C, and separated through a filtration device to obtain 2,5-furandicarboxylic acid. The mole fraction of 2,5-furandicarboxylic acid in the water was 84%, the recovery rate of the amphiphilic catalyst was 96%, and the total yield of 2,5-diformylfuran and 2,5-furandicarboxylic acid was 0.63 mmol.

Embodiment 6

An integrated multi-unit method for co-production of 2,5-diformylfuran and 2,5-furandicarboxylic acid based on a biphasic solvent system comprises the following steps:

Step 1: a reaction solution with a stable water-organic solvent interface was prepared, comprising the following specific steps:

Step 1.1: 3 g of a carbon nanotube was added to a mixed solution of 150 mL of sulphuric acid and 50 mL of nitric acid at room temperature and stirred for 1 h. Then, the solution was stirred in an oil bath at 50 ^{o}C for 4 h to obtain dispersion liquid. The dispersion liquid was diluted, filtered and washed with water for several times to obtain precipitate. The obtained precipitate was dried at 70 ^{o}C for 12 h to obtain a surface-modified carbon nanotube.

Step 1.2: first, at room temperature, the surface-modified carbon nanotube was added to 40 mL of 1 mg mL⁻¹ aqueous palladium chloride solution, subjected to ultrasonic treatment for 5 min and stirred for 6 h to obtain a mixed solution. Then, the mixed solution was dried at 70 ^{o}C for 12 h, and the obtained solid powder was placed into a hydrogen atmosphere, and reduced at a heating rate of 4 ^{o}C/min at 350 ^{o}C for 1.5 h to obtain an amphiphilic catalyst.

Step 1.3: at room temperature, a mixture of 100 mg of 5-hydroxymethylfurfural, 60 mg of the amphiphilic catalyst, 7.5 mL of water and 12.5 mL of cyclohexane was added to a static mixer and processed for 5 min to obtain the reaction solution.

Step 2: 2,5-diformylfuran was synthesized and the organic solvent was recovered, comprising the following specific steps:

Step 2.1: the obtained reaction solution was added to a high pressure batch reactor, purged with oxygen once, subsequently filled with 1.5 MPa of oxygen, heated to 110 ^{o}C, reacted for 4 h, and then cooled to room temperature. The reaction solution was processed in a centrifuge at a rotational speed of 6000 rpm for 6 min to achieve the stratification of the water and the cyclohexane to complete a first reaction, wherein the organic solvent was located in an upper layer and contained the synthesized 2,5-diformylfuran, and a lower layer was the aqueous phase. In the present embodiment, 2,5-diformylfuran was dissolved in the cyclohexane, the amphiphilic catalyst and other products comprising 2,5-furandicarboxylic acid were dispersed in the water phase, and the yield of the product can be determined by HPLC.

Step 2.2: step 1.3 and step 2.1 were repeated, and circular reactions were conducted for five times. Before each circular reaction began, the upper cyclohexane containing 2,5-diformylfuran needed to be transferred and collected at first; subsequently, 100 mg of 5-hydroxymethylfurfural and 12.5 mL of cyclohexane were replenished to the lower water phase; and the mixed solution was added to the static mixer to repeat step 1.3 and step 2.1.

Step 2.3: after the circular reactions were completed, the upper cyclohexane was transferred and collected again; subsequently, the obtained cyclohexane enriched with 2,5-diformylfuran was transferred to a vacuum distillation device; and separation was performed at 10 kPa and 60 ^{o}C. The separated cyclohexane can be recycled in the reaction unit. After all the experimental steps were completed, the cyclohexane can be recovered together with 2,5-diformylfuran. The conversion rate of 5-hydroxymethylfurfural was 100%, the mole fraction of 2,5-diformylfuran in the cyclohexane was 89%, and the recovery rate of the cyclohexane was 88%.

Step 3: 2,5-furandicarboxylic acid was synthesized and the amphiphilic catalyst was recovered, comprising the following specific steps:

Step 3.1: the remaining lower aqueous phase after the separation from step 2.3 was added to the high pressure batch reactor, purged with oxygen once, subsequently filled with 1.5 MPa of oxygen, heated to 110 ^{o}C, and reacted for 24 h, and the intermediate product was completely oxidized into 2,5-furandicarboxylic acid to obtain the reaction solution.

Step 3.2: the reaction solution was processed at a rotational speed of 8000 rpm in the centrifuge for 10 min to obtain the amphiphilic catalyst and an aqueous solution enriched with 2,5-furandicarboxylic acid. The aqueous solution was transferred to a cooling crystallization device, cooled and crystallized at -5 ^{o}C, and separated through a filtration device to obtain 2,5-furandicarboxylic acid. The mole fraction of 2,5-furandicarboxylic acid in the water was 96%, the recovery rate of the amphiphilic catalyst was 84%, and the total yield of 2,5-diformylfuran and 2,5-furandicarboxylic acid was 0.81 mmol.

Reference example 1

Pure water reaction system without multi-unit integration

2 g of a carbon nanotube was added to a mixed solution of 50 mL of nitric acid and 150 mL of sulfuric acid at room temperature and stirred for 0.5 h. Then, the solution was stirred in an oil bath at 60 ^{o}C for 5 h to obtain dispersion liquid. The dispersion liquid was diluted, filtered and washed with water for several times to obtain precipitate. The obtained precipitate was dried at 80 ^{o}C for 12 h to obtain a surface-modified carbon nanotube. At room temperature, the surface-modified carbon nanotube was added to 25 mL of 1 mg mL⁻¹ aqueous ruthenium chloride solution, subjected to ultrasonic treatment for 0.5 min and stirred for 12 h to obtain a mixed solution. Then, the mixed solution was dried at 80 ^{o}C for 12 h, and the obtained solid powder was placed into a hydrogen atmosphere, and reduced at a heating rate of 3 ^{o}C/min at 250 ^{o}C for 2 h to obtain an amphiphilic catalyst.

At room temperature, a mixture of 50 mg of 5-hydroxymethylfurfural, 30 mg of the amphiphilic catalyst and 20 mL of water was added to an ultrasonic emulsifier and processed for 5 min. The obtained reaction solution was added to a high pressure batch reactor, purged with oxygen for three times, subsequently filled with 1 MPa of oxygen, heated to 130 ^{o}C, reacted for 12 h, and then cooled to room temperature. The yield of the product can be determined by HPLC. Results were shown in FIG. 2 and FIG. 3. The conversion rate of 5-hydroxymethylfurfural was 100%, the mole fraction of 2,5-furandicarboxylic acid in the water was 95.6%, and the yield was 0.23 mmol.

Reference example 2

Pure organic reaction system without multi-unit integration

2 g of a carbon nanotube was added to a mixed solution of 50 mL of nitric acid and 150 mL of sulfuric acid at room temperature and stirred for 0.5 h. Then, the solution was stirred in an oil bath at 60 ^{o}C for 5 h to obtain dispersion liquid. The dispersion liquid was diluted, filtered and washed with water for several times to obtain precipitate. The obtained precipitate was dried at 80 ^{o}C for 12 h to obtain a surface-modified carbon nanotube. At room temperature, the surface-modified carbon nanotube was added to 25 mL of 1 mg mL⁻¹ aqueous ruthenium chloride solution, subjected to ultrasonic treatment for 0.5 min and stirred for 12 h to obtain a mixed solution. Then, the mixed solution was dried at 80 ^{o}C for 12 h, and the obtained solid powder was placed into a hydrogen atmosphere, and reduced at a heating rate of 3 ^{o}C/min at 250 ^{o}C for 2 h to obtain an amphiphilic catalyst.

At room temperature, a mixture of 50 mg of 5-hydroxymethylfurfural, 30 mg of the amphiphilic catalyst and 20 mL of toluene was added to an ultrasonic emulsifier and processed for 5 min. The obtained reaction solution was added to a high pressure batch reactor, purged with oxygen for three times, subsequently filled with 1 MPa of oxygen, heated to 130 ^{o}C, reacted for 12 h, and then cooled to room temperature. The yield of the product can be determined by HPLC. Results were shown in FIG. 2 and FIG. 3. The conversion rate of 5-hydroxymethylfurfural was 95.6%, the mole fraction of 2,5-diformylfuran in the toluene was 93.6%, and the yield was 0.35 mmol.

Reference example 3

Biphasic solvent system without multi-unit integration

2 g of a carbon nanotube was added to a mixed solution of 50 mL of nitric acid and 150 mL of sulfuric acid at room temperature and stirred for 0.5 h. Then, the solution was stirred in an oil bath at 60 ^{o}C for 5 h to obtain dispersion liquid. The dispersion liquid was diluted, filtered and washed with water for several times to obtain precipitate. The obtained precipitate was dried at 80 ^{o}C for 12 h to obtain a surface-modified carbon nanotube. At room temperature, the surface-modified carbon nanotube was added to 25 mL of 1 mg mL⁻¹ aqueous ruthenium chloride solution, subjected to ultrasonic treatment for 0.5 min and stirred for 12 h to obtain a mixed solution. Then, the mixed solution was dried at 80 ^{o}C for 12 h, and the obtained solid powder was placed into a hydrogen atmosphere, and reduced at a heating rate of 3 ^{o}C/min at 250 ^{o}C for 2 h to obtain an amphiphilic catalyst.

At room temperature, a mixture of 50 mg of 5-hydroxymethylfurfural, 30 mg of the amphiphilic catalyst, 10 mL of water and 10 mL of toluene was added to an ultrasonic emulsifier and processed for 5 min. The obtained reaction solution was added to a high pressure batch reactor, purged with oxygen for three times, subsequently filled with 1 MPa of oxygen, heated to 130 ^{o}C, reacted for 12 h, and then cooled to room temperature. The reaction solution was processed at a rotational speed of 5000 rpm in the centrifuge for 1 min to achieve the stratification of the water and the toluene. The yield of the product can be determined by HPLC. The conversion rate of 5-hydroxymethylfurfural was 100%, the mole fraction of 2,5-furandicarboxylic acid in the water was 67.5%, and the yield was 0.19 mmol. There is no product in the toluene.

Comparative analysis between reference example 1 and embodiment 3:

Embodiment 3 of the present invention simultaneously achieves the co-production of 2,5-diformylfuran and 2,5-furandicarboxylic acid, and the efficient recovery of the toluene and the catalyst; the mole fraction of 2,5-diformylfuran in the toluene is 98%; the mole fraction of 2,5-furandicarboxylic acid in the water is 94%; the total yield of the two products is as high as 0.77 mmol; and moreover, the recovery rates of the toluene and the catalyst are 85% and 95% respectively. Reference example 1 can only achieve the synthesis of 2,5-furandicarboxylic acid, with a yield of only 0.23 mmol. Due to the biphasic solvent system and the integrated multi-unit method, the present invention demonstrates excellent performance and the feature of easy separation and recovery.

Comparative analysis between reference example 2 and embodiment 3:

Embodiment 3 of the present invention simultaneously achieves the co-production of 2,5-diformylfuran and 2,5-furandicarboxylic acid, and the efficient recovery of the toluene and the catalyst; the mole fraction of 2,5-diformylfuran in the toluene is 98%; the mole fraction of 2,5-furandicarboxylic acid in the water is 94%; the total yield of the two products is as high as 0.77 mmol; and moreover, the recovery rates of the toluene and the catalyst are 85% and 95% respectively. Reference example 2 can only achieve the synthesis of 2,5-diformylfuran, with a yield of only 0.35 mmol. Due to the biphasic solvent system and the integrated multi-unit method, the present invention demonstrates excellent performance and the feature of easy separation and recovery.

Comparative analysis between reference example 3 and embodiment 3:

Embodiment 3 of the present invention simultaneously achieves the co-production of 2,5-diformylfuran and 2,5-furandicarboxylic acid, and the efficient recovery of the toluene and the catalyst; the mole fraction of 2,5-diformylfuran in the toluene is 98%; the mole fraction of 2,5-furandicarboxylic acid in the water is 94%; the total yield of the two products is as high as 0.77 mmol; and moreover, the recovery rates of the toluene and the catalyst are 85% and 95% respectively. Reference example 3 can only achieve the synthesis of 2,5-furandicarboxylic acid, the mole fraction of 2,5-furandicarboxylic acid in the water is only 67.5%, and the yield is only 0.19 mmol. Due to the biphasic solvent system and the integrated multi-unit method, the present invention demonstrates excellent performance and the feature of easy separation and recovery.

The above embodiments only express the implementation of the present invention, and shall not be interpreted as a limitation to the scope of the present invention. It should be noted that, for those skilled in the art, several variations and improvements can also be made without departing from the concept of the present invention, all of which belong to the protection scope of the present invention.

1. An integrated multi-unit method for co-production of 2,5-diformylfuran and 2,5-furandicarboxylic acid based on a biphasic solvent system, which is implemented on the basis of a feedstock pretreatment unit, a reaction unit, a separation unit, a recycling unit and a recovery unit, wherein
in the integrated multi-unit method, first, 5-hydroxymethylfurfural enters the feedstock pretreatment unit, then forms a reaction solution with a stable water-organic solvent interface, passes through the reaction unit to generate 2,5-diformylfuran, 2,5-furandicarboxylic acid and intermediate oxidation products, and then enters the separation unit to achieve the separation of an organic solvent from an aqueous phase, and the separation of 2,5-diformylfuran from the organic solvent in sequence, wherein the organic solvent contains 2,5-diformylfuran and the aqueous phase contains an amphiphilic catalyst, 2,5-furandicarboxylic acid and the intermediate oxidation products; the separated aqueous phase reenters the reaction unit through the recycling unit until the intermediate oxidation products are completely oxidized to 2,5-furandicarboxylic acid, and subsequently, the separation of the amphiphilic catalyst from the aqueous phase, and the separation of 2,5-furandicarboxylic acid from the aqueous phase are achieved in the separation unit; the separated organic solvent can pass through the recycling unit for a next reaction and is recovered after the reaction is completed; finally, the organic solvent, 2,5-diformylfuran, 2,5-furandicarboxylic acid and the amphiphilic catalyst are obtained in the recovery unit;
the method comprises: preparing the reaction solution with a stable water-organic solvent interface; specifically:
1step .1: preparing a surface-modified carbon nanotube;
1step .2: preparing the amphiphilic catalyst;
1-2step 1.2.1: adding the surface-modified carbon nanotube to a metal salt solution, performing ultrasonic treatment and stirring to obtain a mixed solution;
1-2step 1.2.2: drying the mixed solution to obtain solid powder, placing the solid powder into a hydrogen atmosphere and reducing at 100-400 ^{o}C for 1-4 h to obtain the amphiphilic catalyst;
1step .3: building a stable water-organic solvent interface;
at room temperature, adding a mixture of 5-hydroxymethylfurfural, the amphiphilic catalyst obtained in step 1.2, water and the organic solvent to the feedstock pretreatment unit and processing for 2-10 min to obtain the reaction solution with the stable water-organic solvent interface;
a solvent in the metal salt solution is one of water, toluene and cyclohexane, and a solute is metal salt;
the organic solvent is one of toluene, paraxylene, cyclohexane, n-hexane and ethyl acetate.

2. The integrated multi-unit method for co-production of 2,5-diformylfuran and 2,5-furandicarboxylic acid based on a biphasic solvent system according to claim 1, further comprising the following steps:
synthesizing 2,5-diformylfuran and recovering the organic solvent; specifically:
2step .1: adding the reaction solution, obtained in step 1, which has a stable water-organic solvent interface, to a high pressure batch reactor, purging with oxygen, filling with oxygen, heating to 80-160 ^{o}C, reacting for 0.5-5 h, and then cooling to room temperature; processing the reaction solution in a deemulsification device to achieve the stratification of the water and the organic solvent to complete a first reaction, wherein the organic solvent is located in an upper layer and a lower layer is the aqueous phase; the 2,5-diformylfuran is dissolved in the organic solvent, and the amphiphilic catalyst, 2,5-furandicarboxylic acid and the intermediate oxidation products are dispersed in the water;
2-1step 2.2: repeating step 1.3 and step 2.1, and conducting multiple circular reactions, wherein before each circular reaction begins, the upper organic solvent containing 2,5-diformylfuran needs to be transferred and collected at first; subsequently, the same mass of 5-hydroxymethylfurfural and the same mass of organic solvent as those in the first reaction are replenished to the lower water phase; and the mixed solution is delivered to the feedstock pretreatment unit to repeat step 1.3 and step 2.1;
2step .3: after the circular reactions are completed, transferring and collecting the upper organic solvent again; transferring the obtained organic solvent enriched with 2,5-diformylfuran to a vacuum distillation device; and performing separation to obtain the lower aqueous phase and the upper organic solvent, wherein the separated organic solvent is recycled in the reaction unit, and the organic solvent is recovered together with 2,5-diformylfuran;
synthesizing 2,5-furandicarboxylic acid and recovering the amphiphilic catalyst; specifically:
3-2step 3.1: adding the remaining lower aqueous phase after the separation from step 2.3 to the high pressure batch reactor, filling with oxygen, heating to 80-160 ^{o}C again, reacting for 12-36 h, and completely oxidizing the intermediate oxidation products into 2,5-furandicarboxylic acid to obtain the reaction solution;
3step .2: processing the reaction solution in a liquid-solid separation device to obtain the amphiphilic catalyst and an aqueous solution enriched with 2,5-furandicarboxylic acid; transferring the aqueous solution to a cooling crystallization device, cooling and crystallizing, and separating through a filtration device to obtain 2,5-furandicarboxylic acid.

3. The integrated multi-unit method for co-production of 2,5-diformylfuran and 2,5-furandicarboxylic acid based on a biphasic solvent system according to claim 2, wherein in the step 1.1:
the carbon nanotube is added to a mixed strong acid, stirred, uniformly mixed and continued to be stirred under oil bath conditions to obtain dispersion liquid; the dispersion liquid is diluted, filtered and washed with water for several times to obtain precipitate; and the obtained precipitate is dried to obtain the surface-modified carbon nanotube;
the mixed strong acid is a mixed solution of two strong acids, the strong acids are one of hydrochloric acid, sulfuric acid and nitric acid, and the volume ratio of the two strong acids is 3: 1 to 1:3; 0.5-4 g of carbon nanotube is added correspondingly to every 200 mL of the mixed strong acid; the temperature of the continuing stirring under the oil bath conditions is 25-90 ^{o}C, and the time is 2-12 h; and the drying temperature is 50-90 ^{o}C, and the time is 6-24 h.

4. The integrated multi-unit method for co-production of 2,5-diformylfuran and 2,5-furandicarboxylic acid based on a biphasic solvent system according to claim 2, wherein in the step 1.2:
in the step 1.2.1, the metal salt is one of palladium acetate, ruthenium chloride, palladium chloride, chloroplatinic acid, ruthenium acetylacetonate and platinum bis(acetylacetonate); 0.2-3 g of the surface-modified carbon nanotube and 0.02-0.1 g of the metal salt are added correspondingly to every 50 mL of the solvent; the stirring time is 6-24 h;
in the step 1.2.2, the drying temperature is 50-90 ^{o}C, and the time is 6-24 h.

5. The integrated multi-unit method for co-production of 2,5-diformylfuran and 2,5-furandicarboxylic acid based on a biphasic solvent system according to claim 2, wherein in the step 1.3:
the processing time is 2-10 min; the volume ratio of the water to the organic solvent is 0.1-9: 1; and the mass ratio of the amphiphilic catalyst to 5-hydroxymethylfurfural is 0.1-1: 1.

6. The integrated multi-unit method for co-production of 2,5-diformylfuran and 2,5-furandicarboxylic acid based on a biphasic solvent system according to claim 2, wherein in the step 2.1, in the deemulsification device, the reaction solution is processed in the deemulsification device at a rotational speed of 1000-10000 rpm for 1-10 min to achieve the stratification of the water and the organic solvent.

7. The integrated multi-unit method for co-production of 2,5-diformylfuran and 2,5-furandicarboxylic acid based on a biphasic solvent system according to claim 2, wherein in the step 2.3, in the vacuum distillation device, separation conditions are: a pressure of 5-50 kPa and a temperature of 50-100 ^{o}C.

8. The integrated multi-unit method for co-production of 2,5-diformylfuran and 2,5-furandicarboxylic acid based on a biphasic solvent system according to claim 2, wherein the synthesizing 2,5-furandicarboxylic acid and recovering the amphiphilic catalyst are specifically as follows:
in the step 3.1, 0.1-2 MPa of oxygen is filled;
in the step 3.2, in the liquid-solid separation device, the reaction solution is processed at a rotational speed of 5000-18000 rpm in the liquid-solid separation device for 5-15 min; and the temperature of the cooling crystallization device is -30 to 0 ^{o}C.

9. The integrated multi-unit method for co-production of 2,5-diformylfuran and 2,5-furandicarboxylic acid based on a biphasic solvent system according to claim 1, wherein the feedstock pretreatment unit, the reaction unit, the separation unit, the recycling unit and the recovery unit are specifically as follows:
the feedstock pretreatment unit comprises an emulsification device, and the emulsification device is in communication with the reaction unit through a liquid-solid conveying pipeline;
the reaction unit is provided with a high pressure batch reactor, materials passing through the feedstock pretreatment unit undergo chemical reactions in the high pressure batch reactor, and the materials after the reactions are completed pass through the liquid-solid conveying pipeline and enter the separation unit;
the separation unit comprises a deemulsification device, a distillation device, a liquid-solid phase separation device, a cooling crystallization device and a filtration device; the top of the deemulsification device is in communication with the distillation device through a liquid conveying pipeline, and the bottom is connected to the liquid-solid phase separation device through the liquid-solid conveying pipeline; the liquid-solid phase separation device is connected to the cooling crystallization device through the liquid conveying pipeline; the cooling crystallization device is in communication with the filtration device through the liquid-solid conveying pipeline; and the materials passing through the separation unit can enter the recycling unit and the recovery unit through the liquid-solid conveying pipeline.

10. The integrated multi-unit method for co-production of 2,5-diformylfuran and 2,5-furandicarboxylic acid based on a biphasic solvent system according to claim 9, wherein the emulsification device is one of an ultrasonic emulsifier, a high speed stirrer, a high pressure homogenizing emulsifier, and a static mixer.
